(19) United States
(12) Patent Application Publication
(10) Pub. No.: US 2009/0124601 A1
(43) Pub. Date: May 14, 2009

(54) Tartaric Acid Salts of a Dipeptidyl Peptidase-IV Inhibitor

(76) Inventors: Zhiguo J. Song, Edison, NJ (US); Fei Zhang, Edison, NJ (US); Rebecca Leigh Shultz, Maplewood, NJ (US)

Correspondence Address: MERCK AND CO., INC, P O BOX 2000, RAHWAY, NJ, 07065-0907, US

(21) Appl. No.: 11/883,907

(22) PCT Filed: Mar. 24, 2006
(86) PCT No.: PCT/US06/11064

§ 371 Date: Aug. 7, 2007

Related U.S. Application Data
(60) Provisional application No. 60/665,997, filed on Mar. 29, 2005.

(51) Int. Cl. A61K 31/551; C07D 223/10

(52) U.S. Cl. 514/218; 540/529

(57) ABSTRACT

Tartaric acid salts of (3R)-4-[(3R)-3-amino-4-(2,4,5-trifluorophenyl) butanoyl]hexahydro-3-(2,2,2-trifluoroethyl)-2H-1,4-diazepin-2-one are potent inhibitors of dipeptidyl peptidase-IV and are useful for the prevention and/or treatment of non-insulin dependent diabetes mellitus, also referred to as Type 2 diabetes. The invention also relates to crystalline anhydrate forms of the tartaric acid salts as well as a process for their preparation, pharmaceutical compositions containing these novel forms and methods of use for the treatment of Type 2 diabetes.

### FIELD OF THE INVENTION

The present invention relates to particular salts of a dipeptidyl peptidase-IV (DPP-IV) inhibitor. More particularly, the invention relates to hydrogen tartrate salts of (3R)-4-[(3R)-3-amino-4-(2,4,5-trifluorophenyl)butanoyl]hexahydro-3-(2,2,2-trifluoroethyl)-2H-1,4-diazepin-2-one, which is a potent inhibitor of DPP-IV. These novel salts and crystalline anhydrate forms thereof are useful for the treatment and prevention of diseases and conditions for which an inhibitor of DPP-IV is indicated, in particular Type 2 diabetes. The invention further concerns pharmaceutical compositions comprising the hydrogen tartrate salts and their crystalline anhydrate forms which are useful to treat Type 2 diabetes as well as processes for preparing the hydrogen tartrate salts and their crystalline anhydrate forms and their pharmaceutical compositions.

### BACKGROUND OF THE INVENTION

Inhibition of dipeptidyl peptidase-IV (DPP-IV), an enzyme that inactivates both glucose-dependent insulinotropic peptide (GIP) and glucagon-like peptide 1 (GLP-1), represents a novel approach to the treatment and prevention of Type 2 diabetes, also known as non-insulin dependent diabetes mellitus (NIDDM). The therapeutic potential of DPP-IV inhibitors for the treatment of Type 2 diabetes has been reviewed: C. F. Deacon and J. J. Holst, “Dipeptidyl peptidase IV inhibition as an approach to the treatment and prevention of Type 2 diabetes: a historical perspective,” Biochem. Biophys. Res. Commun., 294: 1-4 (2000); K. Augustyns, et al., “Dipeptidyl peptidase IV inhibitors as new therapeutic agents for the treatment of Type 2 diabetes,” Expert. Opin. Ther. Patents, 13: 499-510 (2003); D. J. Drucker, “Therapeutic potential of dipeptidyl peptidase IV inhibitors for the treatment of Type 2 diabetes,” Expert Opin. Investig. Drugs, 12: 87-100 (2003); C. F. Deacon, et al., “Inhibitors of dipeptidyl peptidase IV: a novel approach for the prevention and treatment of Type 2 diabetes?”, Exp. Opin. Investig. Drugs, 13: 1091-1102 (2004); and J. J. Holst, “Treatment of Type 2 diabetes mellitus with agonists of the GLP-1 receptor or DPP-IV inhibitors,” Exp. Opin. Emerg. Drugs, 9: 155-156 (2004).

WO 2004/037169 (published 6 May 2004), assigned to Merck & Co., describes a class of beta-amino hexahydro-1,4-diazepinones, which are potent inhibitors of DPP-IV and therefore useful for the treatment of Type 2 diabetes. Specifically disclosed in WO 2004/037169 is (3R)-4-[(3R)-3-amino-4-(2,4,5-trifluorophenyl)butanoyl]hexahydro-3-(2,2,2-trifluoroethyl)-2H-1,4-diazepin-2-one. Pharmaceutically acceptable salts of this compound are generically encompassed within the scope of WO 2004/037169.

However, there is no specific disclosure in the above reference of the newly discovered monobasic hydrogen tartrate salts of (3R)-4-[(3R)-3-amino-4-(2,4,5-trifluorophenyl)butanoyl]hexahydro-3-(2,2,2-trifluoroethyl)-2H-1,4-diazepin-2-one of structural formula I below.

### SUMMARY OF THE INVENTION

The present invention is concerned with novel hydrogen tartrate salts of the dipeptidyl peptidase-IV (DPP-IV) inhibitor (3R)-4-[(3R)-3-amino-4-(2,4,5-trifluorophenyl)butanoyl]hexahydro-3-(2,2,2-trifluoroethyl)-2H-1,4-diazepin-2-one and, in particular, crystalline anhydrate forms thereof. The crystalline hydrogen tartrate salts of the present invention have advantages in the preparation of pharmaceutical compositions of (3R)-4-[(3R)-3-amino-4-(2,4,5-trifluorophenyl)butanoyl]hexahydro-3-(2,2,2-trifluoroethyl)-2H-1,4-diazepin-2-one, such as ease of processing, handling, and dosing. In particular, they exhibit improved physicochemical properties, such as solubility, stability to stress, and rate of solution, rendering them particularly suitable for the manufacture of various pharmaceutical dosage forms. The invention also concerns pharmaceutical compositions containing the novel hydrogen tartrate salts and crystalline anhydrate forms thereof as well as methods for using them as DPP-IV inhibitors, in particular, for the prevention or treatment of Type 2 diabetes.

### BRIEF DESCRIPTION OF THE DRAWINGS

FIG. 1 is a characteristic X-ray diffraction pattern of the crystalline anhydrate form of the L-hydrogen tartrate salt of structural formula II of the present invention.

FIG. 2 is a carbon-13 cross-polarization magic-angle spinning (CPMAS) nuclear magnetic resonance (NMR) spectrum of the crystalline anhydrate form of the L-hydrogen tartrate salt of structural formula II of the present invention.

FIG. 3 is a fluorine-19 magic-angle spinning (MAS) nuclear magnetic resonance (NMR) spectrum of the crystalline anhydrate form of the L-hydrogen tartrate salt of structural formula II of the present invention.

FIG. 4 is a typical differential scanning calorimetry (DSC) curve of the crystalline anhydrate form of the L-hydrogen tartrate salt of structural formula II of the present invention.

### DETAILED DESCRIPTION OF THE INVENTION

This invention provides new monobasic hydrogen tartrate salts of (3R)-4-[(3R)-3-amino-4-(2,4,5-trifluorophenyl)butanoyl]hexahydro-3-(2,2,2-trifluoroethyl)-2H-1,4-diazepin-2-one of the following structural formula I:

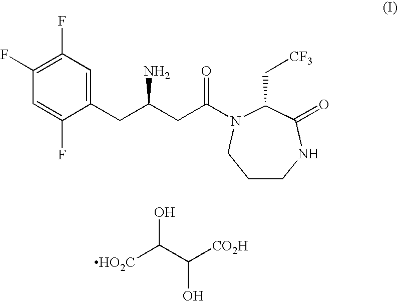

In particular, the instant invention provides a crystalline anhydrate form of the hydrogen tartrate salts of formula I.

One embodiment of the present invention provides the L-hydrogen tartrate salt of (3R)-4-[(3R)-3-amino-4-(2,4,5-trifluorophenyl)butanoyl]hexahydro-3-(2,2,2-trifluoroethyl)-2H-1,4-diazepin-2-one of structural formula II:

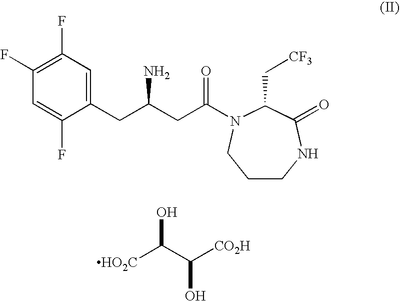

and a crystalline anhydrate form thereof.

A second embodiment of the present invention provides the D-hydrogen tartrate salt of (3R)-4-[(3R)-3-amino-4-(2,4,5-trifluorophenyl)-butanoyl]hexahydro-3-(2,2,2-trifluoroethyl)-2H-1,4-diazepin-2-one of structural formula III:

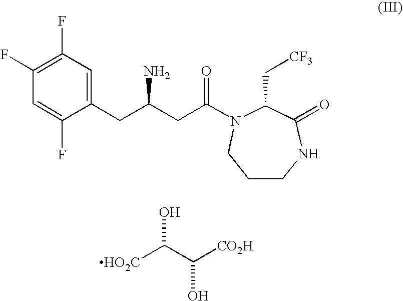

and a crystalline anhydrate form thereof.

More specifically, the hydrogen tartrate salts of the present invention are comprised of one molar equivalent of mono-protonated (3R)-4-[(3R)-3-amino-4-(2,4,5-trifluorophenyl)butanoyl]hexahydro-3-(2,2,2-trifluoroethyl)-2H-1,4-diazepin-2-one cation and one molar equivalent of hydrogen tartrate anion.

In a further embodiment of the present invention, the hydrogen tartrate salts of structural formulae I-III are in the form of a crystalline anhydrate.

A further embodiment of the present invention provides the hydrogen tartrate drug substance of structural formulae I-III that comprises a crystalline anhydrate form present in a detectable amount. By “drug substance” is meant the active pharmaceutical ingredient. The amount of crystalline anhydrate form in the drug substance can be quantified by the use of physical methods such as X-ray powder diffraction, solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy. In a class of this embodiment, about 5% to about 100% by weight of the crystalline anhydrate form is present in the drug substance. In a second class of this embodiment, about 10% to about 100% by weight of the crystalline anhydrate form is present in the drug substance. In a third class of this embodiment, about 25% to about 100% by weight of the crystalline anhydrate form is present in the drug substance. In a fourth class of this embodiment, about 50% to about 100% by weight of the crystalline anhydrate form is present in the drug substance. In a fifth class of this embodiment, about 75% to about 100% by weight of the crystalline anhydrate form is present in the drug substance. In a sixth class of this embodiment, substantially all of the hydrogen tartrate salt drug substance is the crystalline anhydrate form of the present invention, i.e., the hydrogen tartrate salt drug substance is substantially phase pure crystalline anhydrate form.

The crystalline hydrogen tartrate salts of the present invention exhibit pharmaceutic advantages over the free base and the previously disclosed hydrochloride salt (WO 04/037169) in the preparation of a pharmaceutical drug product containing the pharmacologically active ingredient. In particular, the enhanced chemical and physical stability of the crystalline hydrogen tartrate salt anhydrate forms constitute advantageous properties in the preparation of solid oral dosage forms containing the pharmacologically active ingredient. The crystalline hydrogen tartrate salt anhydrate forms are single, high-melting forms and are non-hygroscopic.

The hydrogen tartrate salts of the present invention and their crystalline anhydrate forms, which exhibit potent DPP-IV inhibitory properties, are particularly useful for the prevention or treatment of Type 2 diabetes.

Another aspect of the present invention provides a method for the prevention or treatment of clinical conditions for which an inhibitor of DPP-IV is indicated, which method comprises administering to a patient in need of such prevention or treatment a prophylactically or therapeutically effective amount of a hydrogen tartrate salt of structural formula I-III or a crystalline anhydrate form thereof. Such clinical conditions include diabetes, in particular Type 2 diabetes.

The present invention also provides the use of a hydrogen tartrate salt of structural formula I-III or a crystalline anhydrate form thereof for the manufacture of a medicament for the prevention or treatment of clinical conditions for which an inhibitor of DPP-IV is indicated.

The present invention also provides pharmaceutical compositions comprising a hydrogen tartrate salt of structural formula I-III or a crystalline anhydrate form thereof in association with one or more pharmaceutically acceptable carriers or excipients. In one embodiment the pharmaceutical compositions comprise a therapeutically effective amount of the active pharmaceutical ingredient in admixture with pharmaceutically acceptable excipients wherein the active pharmaceutical ingredient comprises a detectable amount of a crystalline anhydrate form of the present invention. In a second embodiment the pharmaceutical compositions comprise a therapeutically effective amount of the active pharmaceutical ingredient in admixture with pharmaceutically acceptable excipients wherein the active pharmaceutical ingredient comprises about 5% to about 100% by weight of a crystalline anhydrate form of the present invention. In a class of this second embodiment, the active pharmaceutical ingredient in such compositions comprises about 10% to about 100% by weight of a crystalline anhydrate form. In a second class of this embodiment, the active pharmaceutical ingredient in such compositions comprises about 25% to about 100% by weight of a crystalline anhydrate form. In a third class of this embodiment, the active pharmaceutical ingredient in such compositions comprises about 50% to about 100% by weight of a crystalline anhydrate form. In a fourth class of this embodiment, the active pharmaceutical ingredient in such compositions comprises about 75% to about 100% by weight of a crystalline anhydrate form. In a fifth class of this embodiment, substantially all of the active pharmaceutical ingredient is a crystalline hydrogen tartrate salt anhydrate of the present invention, i.e., the active pharmaceutical ingredient is substantially phase pure crystalline hydrogen tartrate salt anhydrate.

The compositions in accordance with the invention are suitably in unit dosage forms such as tablets, pills, capsules, powders, granules, sterile solutions or suspensions, metered aerosol or liquid sprays, drops, ampoules, auto-injector devices or suppositories. The compositions are intended for oral, parenteral, intranasal, sublingual, or rectal administration, or for administration by inhalation or insufflation. Formulation of the compositions according to the invention can conveniently be effected by methods known from the art, for example, as described in Remington's Pharmaceutical Sciences, 17^{th} ed., 1995.

The dosage regimen is selected in accordance with a variety of factors including type, species, age, weight, sex and medical condition of the patient; the severity of the condition to be treated; the route of administration; and the renal and hepatic function of the patient. An ordinarily skilled physician, veterinarian, or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 200 mg of active ingredient. Intravenously, the most preferred doses will range from about 0.1 to about 10 mg/kg/minute during a constant rate infusion. Advantageously, the crystalline anhydrate forms of the present invention may be administered in a single daily dose, or the total daily dosage may be administered in divided doses of two, three or four times daily. Furthermore, the crystalline anhydrate forms of the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the present invention, the hydrogen tartrate salts and their crystalline anhydrate forms herein described in detail can form the active pharmaceutical ingredient, and they are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug component can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The hydrogen tartrate salts of structural formula I-III and their crystalline anhydrate forms have been found to possess a high solubility in water, rendering them especially amenable to the preparation of formulations, in particular intranasal and intravenous formulations, which require relatively concentrated aqueous solutions of active ingredient.

According to a further aspect, the present invention provides a process for the preparation of the hydrogen tartrate salts of formula I-III, which process comprises reacting (3R)-4-[(3R)-3-amino-4-(2,4,5-trifluorophenyl)butanoyl]hexahydro-3-(2,2,2-trifluoroethyl)-2H-1,4-diazepin-2-one hydrochloride salt of structural formula IV below:

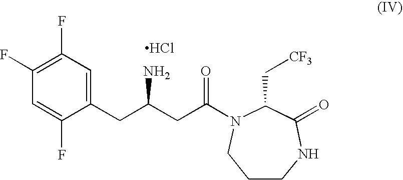

with approximately one to two equivalents of choline bicarbonate in the presence of one to two equivalents of the appropriate form of tartaric acid in a suitable C₁-C₅ alkanol, such as methanol, ethanol, isopropyl alcohol (IPA), and isoamyl alcohol (IAA) or aqueous C₁-C₅ alkanol. The reaction is carried out at a temperature range of about 25° C. to about 80° C. The crystalline hydrogen tartrate salt anhydrate is obtained by crystallization from the aqueous C₁-C₅ alkanol solution which can be accelerated by the addition of a small amount of seed crystals. In one embodiment of this process, the aqueous alkanol is aqueous isopropanol (IPA).

The following non-limiting Examples are intended to illustrate the present invention and should not be construed as being limitations on the scope or spirit of the instant invention.

The starting compound of structural formula IV can be prepared by the procedure detailed in Example 1 below.

Compounds described herein may exist as tautomers such as keto-enol tautomers. The individual tautomers as well as mixtures thereof are encompassed with compounds of structural formula I-III.

The term “% enantiomeric excess” (abbreviated “ee”) shall mean the % major enantiomer less the % minor enantiomer. Thus, an 80% enantiomeric excess corresponds to formation of 90% of one enantiomer and 10% of the other. The term “enantiomeric excess” is synonymous with the term “optical purity.”

The term “enantiomerically enriched” shall mean that a compound of structural formula I-III is obtained by the process of the present invention with an enantiomeric excess of the desired (R)-enantiomer greater than 70% over the (S)-enantiomer. In one embodiment a compound of formula I-III having the (R)-configuration is obtained with an ee greater than 80%. In a class of this embodiment the (R)-enantiomer is obtained with an ee greater than 90%. In a subclass of this class the (R)-enantiomer is obtained with an ee greater than 95%.

The term “% diastereomeric excess” (abbreviated “de”) shall mean the % major diastereomer less the % minor diastereomer. Thus, an 80% diastereomeric excess corresponds to formation of 90% of one diastereomer and 10% of the other.

The term “diastereomeric ratio” (abbreviated “dr”) shall mean the % major diastereomer divided by the % minor diastereomer. Thus, a diastereomeric ratio of 19 corresponds to formation of 95% of one diastereomer and 5% of the other.

The term “enantioselective” shall mean a reaction in which one enantiomer is produced (or destroyed) more rapidly than the other, resulting in the predominance of the favored enantiomer in the mixture of products.

The term “diastereoselective” shall mean a reaction in which one diastereomer is produced (or destroyed) more rapidly than the other, resulting in the predominance of the favored diastereomer in the mixture of products.

By “L-tartaric acid” is meant the dextrorotatory enantiomeric form of tartaric acid. By “D-tartaric acid” is meant the levorotatory enantiomeric form of tartaric acid.

### EXAMPLE 1

### (3R)-4-[(3R)-3-amino-4-(2,4,5-trifluoropheyl)butanoyl]hexahydro-3-(2,2,2-trifluoroethyl)-2H-1,4-diazepin-2-one L-hydrogen tartrate anhydrate

### Step A: Ethyl 2-amino-4,4,4-trifluorobutanoate hydrochloride salt (1-2)

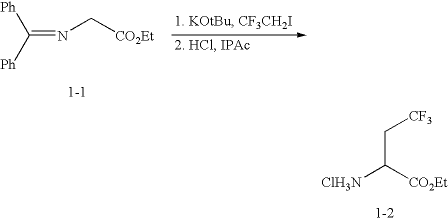

To a 100-L jacketed vessel equipped with overhead stirrer, nitrogen inlet, vacuum inlet, and thermocouple was charged 35 L of DMF and cooled to −20° C. The vessel was purged by nitrogen gas. Potassium t-butoxide (3.11 kg, 27.8 mol) was added with vigorous stirring. The mixture was aged for 5 min to allow dissolution of the solid. The glycine imine 1-1 (7.00 kg, 26.2 mol) was added. Air was removed from the reaction vessel using three vacuum/nitrogen fill cycles. 2,2,2-Trifluoroethyl iodide was charged into a 5-L round bottom flask. The iodide was transferred into the stirring enolate solution in portions using residual vacuum. The mixture was aged at 0° C. for 5 h and then slowly warmed to room temperature over 1 h and held overnight. Half of the reaction mixture was transferred into a 100-L extractor containing 18 L of 5% ammonium chloride solution and 35 L of isopropyl acetate (IPAc) at 10° C. The mixture was vigorously stirred, the layers allowed to settle, and the lower aqueous layer separated. The organic layer was washed three times with 18 L of 2% sodium chloride solution. The process was repeated with the second half of the reaction mixture. The combined organic layers were concentrated in a 100-L round bottom flask attached to a batch concentrator at 20-25° C., 28-29 in Hg. Concentrated hydrochloric acid (2.7 L, 32.7 mol) was added. The batch was heated to 50° C. and aged for 30 min. The batch was then concentrated at 55-60° C., 21-23 in Hg to 35 L total volume. The batch was then solvent switched into IPAc with constant feed distillation at 55-60° C. A total of 50 L of IPAc was flushed through. The slurry was allowed to slowly cool to room temperature. The solid was isolated by filtration. The cake was washed with IPAc, 7 L displacement wash, 7 L slurry wash, and 5 L displacement wash. The cake was dried on the filter under nitrogen. The trifluoroethyl amino ester 1-2 was obtained as an off-white solid.

### Step B: 2-[(2-Cyanoethyl)amino]-4,4,4-trifluorobutanoic acid (1-3)

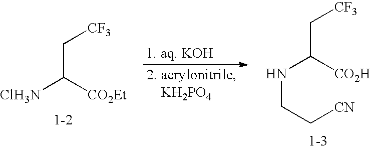

To 100-L cylindrical vessel equipped with coiling coils, thermocouple, nitrogen inlet, and vacuum inlet was charged water (35.7 L) and potassium hydroxide (4.38 kg, 67.7 mol) resulting in an exotherm to 36° C. The mixture was cooled to 12° C. (cooling coils set at −20° C.), and trifluoroethyl aminoester HCl 1-2 (7.14 kg, 32.2 mol) was) charged over 30 min while maintaining the temperature under 15° C. The coiling coils were set to 20° C., and after the ice on the coils melted, the coils and sides of the vessel were rinsed with water (2.0 L). Air was removed from the vessel by vacuum/nitrogen cycling. The reaction was aged for 1 h at 15-20° C. The reaction solution was transferred through a 20 μm and then a 5 μm in-line filter to a 100-L, 4-neck round bottom flask equipped with overhead stirring, thermocouple, nitrogen inlet, and vacuum inlet. Potassium monophosphate (1.17M solution, 3.80 L) was charged in portions to pH 9.84. Air was removed from the vessel by vacuum/nitrogen cycling, and then acrylonitrile (3.18 L, 48.3 mol) was) charged in one portion at room temperature with a nitrogen sweep. Air was again removed from the vessel by vacuum/nitrogen cycling, and the reaction was aged at room temperature overnight. With cooling from a cool water bath, concentrated hydrochloric acid (0.18 L) was charged dropwise via addition funnel over 15 min to the reaction solution to induce seeding. The resulting slurry was aged for 40 min to develop a seed bed. The remaining concentrated hydrochloric acid (3.17 L) was charged via addition funnel over 1.75 h maintaining the temperature below 30° C. The resulting white slurry was aged for 1 h. The solids were isolated via filtration using a 23.5 inch diameter filter pot. The cake was washed twice with 13.0 L water followed by a displacement wash with 14.2 L MeCN. The cake was dried on the filter under nitrogen. The Michael adduct 1-3 was obtained as a white, free flowing solid.

### Step C: 2-[(3-Aminopropyl)amino]-4,4,4-trifluorobutanoic acid (1-4)

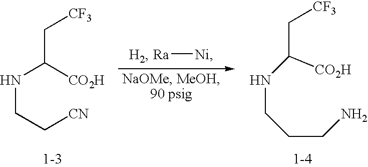

To a slurry of 2.7 Kg (12.85 mol) of nitrile 1-3 in 11.5 L of MeOH were charged 4.17 Kg (19.31 mol) of 25 wt % NaOMe solution in MeOH. All solids dissolved after 20 min of stirring. Raney nickel-2800 slurry in water (23 wt %, 625 g) was charged to the solution and the vessel charged with hydrogen at 90 psig at 25° C., in a stirred autoclave. After 18 h, the catalyst was filtered over Celite and washed with 6.5 L of MeOH.

### Step D: 3-(2,2,2-Trifluoroethyl)-1,4-diazepan-2-one (1-5)

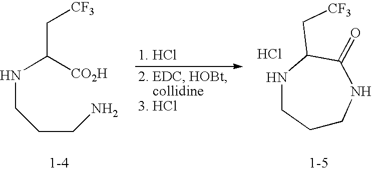

To 59.0 Kg of the methanolic solution from Step C containing 5.27 Kg of diaminoacid 1-4 (24.6 mol) were charged 3.8 Kg of concentrated hydrochloric acid (37 wt %, 38.7 mol). The temperature rose to 33° C. An ice bath was used to cool the resulting slurry to 20° C. After combining the filtrate and washings from the previous step, the concentration of diaminoacid 1-4 in solution was around 90 mg/g (about 10 L/Kg of 1-4). HOBt (665 g, 4.92 mol, 20 mol %) and collidine (596 g, 4.92 mol, 20 mol %) were then added. The slurry was aged at 20° C. for 10 min and EDC (4.99 Kg, 26.0 mol) was charged over 30 min. A slight exotherm of 6° C. was registered. After aging overnight, the crude reaction mixture was filtered through a 10-15 micron pore size filter to remove the solids in suspension. A total of 18.7 Kg of solution was obtained after the filtration. The solution was concentrated at reduced pressure to 11 Kg total weight. 5-6 N HCl in IPA was added to this solution until pH of 3 was obtained (5.3 L). The temperature was kept below 39° C. with an ice bath. The slurry was aged overnight at 20° C. The solids were filtered and washed with 9 Kg of IPA and dried in the filter pot.

### Step E: 2,2-Dimethyl-5-[(2,4,5-trifluorophenyl)acetyl]-1,3-dioxan-4,6-dione (1-6)

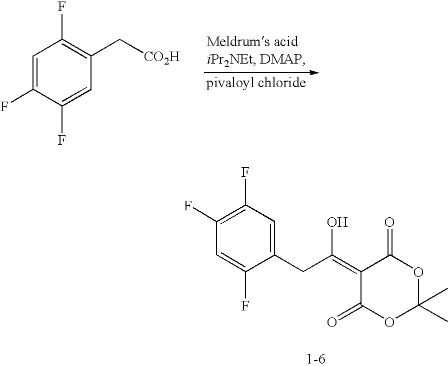

Trifluorophenylacetic acid (3.5 kG, 18.4 mol), Meldrum's acid (2.92 kG, 20.25 mol), and DMAP (225 g, 1.84 mol) were charged into a 72 L three-neck flask. MeCN (14 L) was added in one portion at room temperature to dissolve the solids. iPr₂NEt (7.06 L, 40.5 mol) was added in one portion at room temperature. Pivaloyl chloride (2.5 L, 20.25 mol) was then added dropwise over 1 to 2 h while the reaction temperature was maintained below 55° C. The reaction was then aged at 50° C. for 2-3 h. The reaction was cooled to 20° C. and 7 L of 17.7 wt % aqueous phosphoric acid was charged to homogeneous solution over 1 h. The product crystallized out of solution and slurry was aged 1 h. Then an additional 21 L of 17.7 wt % phosphoric acid was charged and final pH of aqueous layer was 2.5. The slurry was filtered at ambient temperature and the mother liquors recycled to remove all solids from the flask. The cake was washed with 15 L of 2:3 MeCN/H₂O and the wet calke stirred and then filtered. The cake was washed an additional two times with 15 L of 2:3 MeCN/H₂O and filtered. The wet cake was then dried in vacuum oven at 40° C. for up to 5 d to afford Meldrum's adduct 1-6.

¹H-NMR (400 MHz, CDCl₃): δ 15.50 (s, 1H), 7.14 (m, 1H), 6.96 (m, 1H), 4.45 (s, 2H), 1.76 (s, 6H) ppm;

¹³C-NMR (100 MHz, CDCl₃): δ 192.76, 170.66, 160.42, 156.47 (ddd, J_{CF}=245.7, 9.6, 2.4 Hz), 149.79 (ddd, J_{CF}=251.4, 14.5, 12.0 Hz), 146.90 (ddd, J_{CF}=244.9, 12.0, 3.2 Hz), 119.40 (dd, J_{CF}=19.3, 5.6 Hz), 117,41, 105.63, 91.99, 34.59, 27.06 ppm.

### Step F: 4-[3-Oxo-4-(2,4,5-trifluorophenyl)butanoyl]-3-(2,2,2-trifluoroethyl)-1,4-diazepan-2-one (1-7)

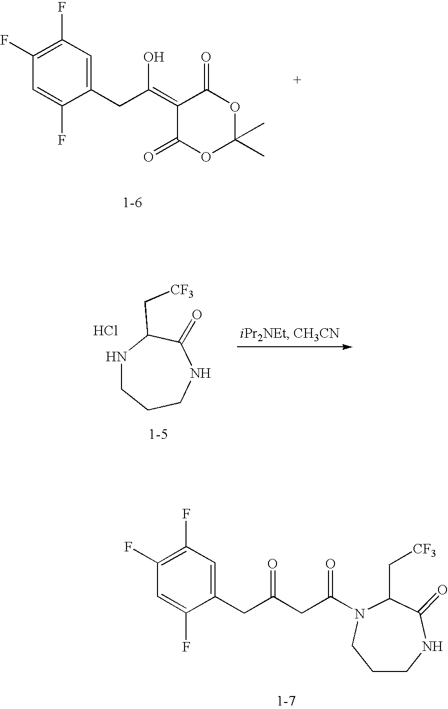

Meldrum's adduct 1-6 (5.62 kG, 17.8 mol) and 18 L of MeCN was charged to 100-L cylindrical vessel equipped with bubbler. 2.7 L (15.48 mol) of iPr₂NEt was then charged to slurry. Diazapinone HCl 1-5 (3.6 kG, 15.48 mol) was then charged to the homogeneous solution in one portion followed by 18 L of MeCN to rinse solids from side of flask. The slurry was heated to 40° C. and aged for at least 12 h. The reaction was then cooled to ambient temperature and 25 L of MTBE was charged to reaction followed by 14 L of water. The aqueous layer was discarded. The organics were washed with 25 L of 7 wt % NaHCO₃ and aqueous layer was discarded. The organics were washed with 25 L of 20 wt % NaCl and aqueous layer was discarded. The organics were then solvent switched into isopropanol for the subsequent step.

### Step G: 4-[(2Z)-3-Amino-4-(2,4,5-trifluorophenyl)but-2-enoyl]-3-(2,2,2-trifluoroethyl)-1,4-diazepan-2-one (1-8)

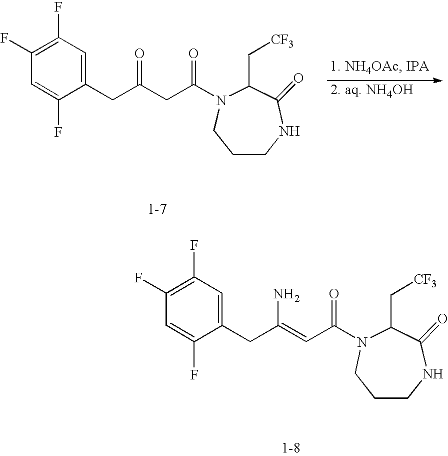

5.3 kg (12.92 mol) of ketoamide 1-7 in MTBE layers were charged into a clean 72-L round-bottomed flask. During this charge, the MTBE was distilled away, maintaining an internal volume of about 26.5 L (5 L/kcg). After completion of the charge and a rinse with about 0.5 L isopropanol, the solution was solvent-switched at the same constant volume to isopropanol, followed by azeotropic drying with IPA until Karl Fisher test was less than 5000 (about 75 L total volume solvent removed, 60 L IPA charged). To the heterogeneous mixture of ketoamide 1-7 in IPA was added 3.98 kg (51.67 mol) of ammonium acetate. The reaction was heated to 45° C. and aged 3 h. The reaction mixture was then cooled to room temperature, then quenched over 15 min with aqueous ammonium hydroxide (14.8M, 1.73 kg, 25.84 mol) while keeping the internal temperature below 30° C. Enamine amide 1-8 was further crystallized by the slow addition of 26.5 L (5 L/kg) of water over 2 h. The crystallization mixture was aged at room temperature overnight. The batch was then filtered, slurry washed once with 10.6 L of 50/50 IPA/water (2 L/kg), displacement washed once with 10.6 L of 50/50 IPA/water (2 L/kg), then dried under active nitrogen overnight.

### Step H: 4-[(3R)-3-(tert-Butyloxycarbonylamino)-4-(2,4,5-trifluorophenylbutanoyl]-3-(2,2,2-trifluoroethyl)-1,4-diazepin-2-one (1-9 and 1-10)

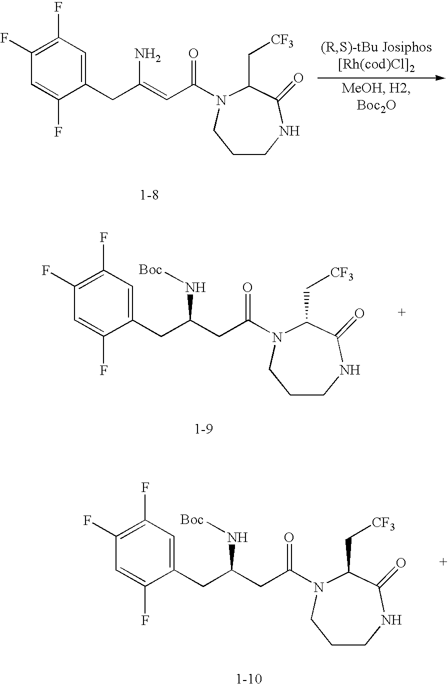

To a 10-gallon autoclave was charged as a slurry mixture using 21.6 L of methanol, 4.4 kg (10.8 mol) of enamine amide 1-8, 2.47 kg (11.3 mol) of di-t-butyl dicarbonate, and [Rh(cod)Cl]₂ (5.33 g, 10.8 mmol). The substrate mixture was degassed with 5 pump-purge cycles. Under an inert atmosphere, the (R,S)-t-butyl Josiphos (12.3 g, 22.6 mol) was suspended in 0.4 L of degassed methanol in an inert transfer device. A rinse of 0.1 L was charged to the second container in the device. The ligand was transferred under inert conditions to the substrate/metal slurry in the autoclave. The entire mixture was purged twice. The autoclave was subjected to 100 psig of hydrogen gas at 20° C. for 18 h. The vessel was drained and the vessel was rinsed with 5-6 volumes of methanol. Chiral HPLC assay indicated that the stereogenic center to which the tert-butoxycarbonylamino group is attached was 98% optically pure and the ratio of 1-9 to 1-10 was about 1:1. The slurry was used directly in the next step.

### Step I: (3R)-4-[(3R)-3-(tert-Butyloxycarbonylamino)-4-(2,4,5-trifluorophenylbutanoyl]-3-(2,2,2-trifluoroethyl-1,4-diazepin-2-one (1-9)

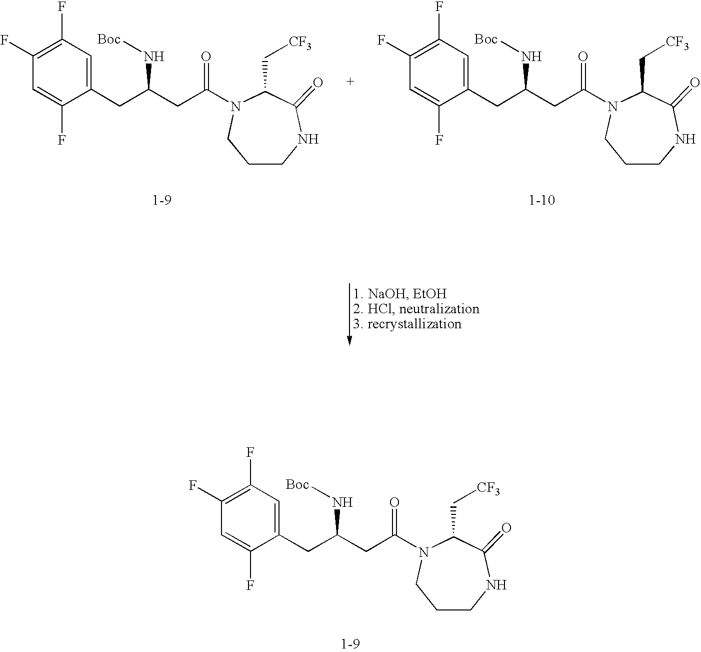

Into a 100 L Buchi vessel was charged the slurry of 5.44 kg hydrogenation product mixture in about 24 L MeOH. The mixture was concentrated to about 20 L and then solvent-switched to EtOH with about 20 L of EtOH. The whole process took approximately two h and the resulting slurry was diluted with EtOH to about 35 L. After diluting the batch with EtOH to a total volume of about 55 L, NMR indicated that 5.9 wt % of MeOH was present in the final solvent system.

The ethanolic slurry was slowly heated to 70° C. and total dissolution occurred at about 68° C. The resulting clear yellowish solution was slowly cooled at a rate of 5° C./30 min and seeded. The cooling rate was kept constant at 5° C./30 min (significant crystallization was observed at 60° C.) until the batch reached 40° C. To the vigorously agitated slurry at 40° C. there was slowly charged 1N ethanolic NaOH prepared by mixing 2.7 L of EtOH, 81 g of water, and 1630 mL of ethanolic sodium ethoxide (purchased as 21 wt % solution and titrated to be 2.76 M). The slurry was further cooled to room temperature and agitated at this temperature for one h. To ensure complete epimerization, the slurry was chilled to 0° C. within two h and agitated at 0° C. overnight.

While maintaining the temperature at about 0° C., the basic slurry was neutralized with 1N ethanolic HCl (prechilled to 0° C.) prepared by diluting 395 mL aqueous HCl (purchased as 37 wt % solution and titrated to be 11.39 M) with EtOH to 4.5 L. The addition rate was carefully controlled and the whole neutralization process, accompanied by frequent pH checking, took about 30 min. The pH of the slurry was tested to be about 5 after addition of about 95% amount of prepared HCl solution. Another 40 L of EtOH was used to rinse off the splash on the wall of reaction vessel.

The neutral slurry was gradually heated to 70° C. and total dissolution of 1-9 and 1-10 was observed at 68° C. The cloudy solution was slowly cooled at a rate of 5° C./30 min and seeded with about 10 g of 1-9 at 65° C. The cooling rate was kept at 5° C./30 min (significant crystallization occurred at about 60° C.) until the batch reached 20° C. The thick slurry was brought to 0° C. and stirred at this temperature overnight.

The slurry was filtered and washed with 15 L EtOH (displacement wash), 2×15 L EtOH and 2×15 L water followed by 15 L EtOH. The wet cake was then allowed to stand under high vacuum suction (with N₂ bag) overnight, spread into four glass trays and further dried in oven (about 400 Torr, 40° C., with N₂ flow) for 72 h. Among four trays of product dried in oven, one tray still contained about 4% water as tested by Karl Fisher and the rest of three trays all contained less than 0.8 wt % water. The tray containing wet product was further dried in oven (about 400 Torr, 40° C., with N₂ flow) for 24 h and Karl Fisher-tested again (less than 0.5 wt % water). The product in four trays was combined. HPLC assay of final product indicated 99.5% pure 1-9.

### Chiral HPLC Conditions:

Column: Chiralpac AD-H (size: 4.6×250 mm) 5 μm packing
Eluent: 0-15 min: 80% Heptane (with 0.1% DEA)/20% EtOH (with 0.1% DEA)
Flow rate: 1.0 mL/min
Temperature: 40° C.
Detector: UV detector @ 254 nm
Injection: 5 μL

### Retention Times:

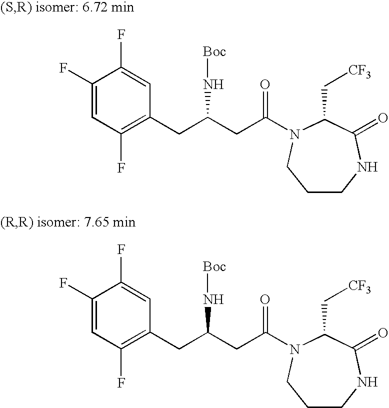

(S,S) isomer: 8.20 min

### Step J: (3R)-4-[(3R)-3-amino-4-(2,4,5-trifluorophenyl)butanoyl]-3-(2,2,2-trifluoroethyl-1,4-diazepin-2-one L-hydrogen tartrate anhydrate (1-11)

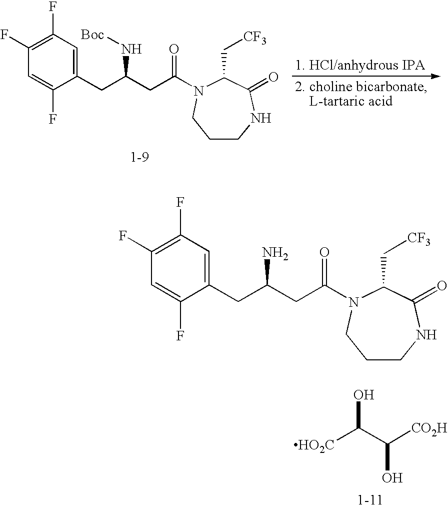

Into a 100 L cylindrical vessel equipped with reflux condenser, thermocouple and nitrogen inlet was charged 14 L IPA. Compound 1-9 (3.78 kg) isolated from the previous epimerization Step I was portionwise added to the vessel and another 12.5 L of IPA was used to rinse off residual 1-9 on the funnel. Upon the completion of addition of starting material, HCl in IPA (2.24 L, purchased as 5 M solution in IPA and titrated to be 4.95 M) was charged to the slurry with sufficient stirring and the batch was heated to 75° C. Although the temperature rose quickly from room temperature to about 70° C., the internal temperature of the reaction was in the range of 70-75° C. for over two h and the thick white slurry turned into a slightly turbid yellow solution during this period due to presence of insoluble NaCl. The reaction mixture was cooled 10-20° C. To the batch was added 14.1 L water and 2055 g L-tartaric acid. This solution was filtered through an 1 micron in-line filter into the 100 L reaction vessel. The resulting filtrate was then diluted to 62 L. To the reaction mixture was slowly added 2.29 L 75% choline bicarbonate (5.23 M, 1.75 eq). Carbon dioxide bubbles were observed from the reaction mixture, and solids precipitated out. The batch was heated to near reflux (75-80° C.) to dissolve all solids. Then 20 L IPA was added while maintaining the batch at 72-80° C. The batch was then seeded at 72° C. (20 g), aged for 35 min (72-75° C.) and then cooled slowly to 15° C. at a cooling rate of 0.1° C./min. The batch was stirred overnight at 15-20° C. The slurry was filtered and the solid product was washed five times with 8 L of IPA. The white solid was then dried in a vacuum oven at 40° C. until no more weight loss (1-2 d). A total of 3.91 kg product was obtained. HPLC assay indicated 99.5% 1-11 and 0.3% of the (R,S)-diastereo Chiral assay indicated 100% ee. [α]_{D}-72° (80/20 v/v water/MeCN, 1% wt/v, 405 nm).

### Reversed Phase HPLC Conditions:

### Column:

YMC ODS-AQ (size: 4.6×250 mm)
ODS-AQ-303-5 5 μm packing and 120A pore

### Eluent:

| | |
|---|---|
| 0-10 min: | 10% MeCN/90% aqueous 0.1% H₃PO₄ |
| 10-20 min: | 30% MeCN/70% aqueous 0.1% H₃PO₄ |
| Flow rate: | 1.5 mL/min constant |
| Temperature: | Ambient |
| Detector: | UV detector @ 210 nm |
| Injection: | 5 μL |
| Retention times: | Compound 1-11: 6.87 min; |
| | R,S-isomer of 1-11: 7.08 min; |
| | Compounds 1-9 and 1-10: 11.2 min. |

X-ray powder diffraction studies are widely used to characterize molecular structures, crystallinity, and polymorphism. The X-ray powder diffraction pattern of the crystalline hydrogen tartrate anhydrate was generated on a Philips Analytical X'Pert PRO X-ray Diffraction System with PW3040/60 console. A PW3373/00 ceramic Cu LEF X-ray tube K-Alpha radiation was used as the source.

The crystal form of the solids was shown to be anhydrate by X-ray powder diffraction and thermogravimetric analysis.

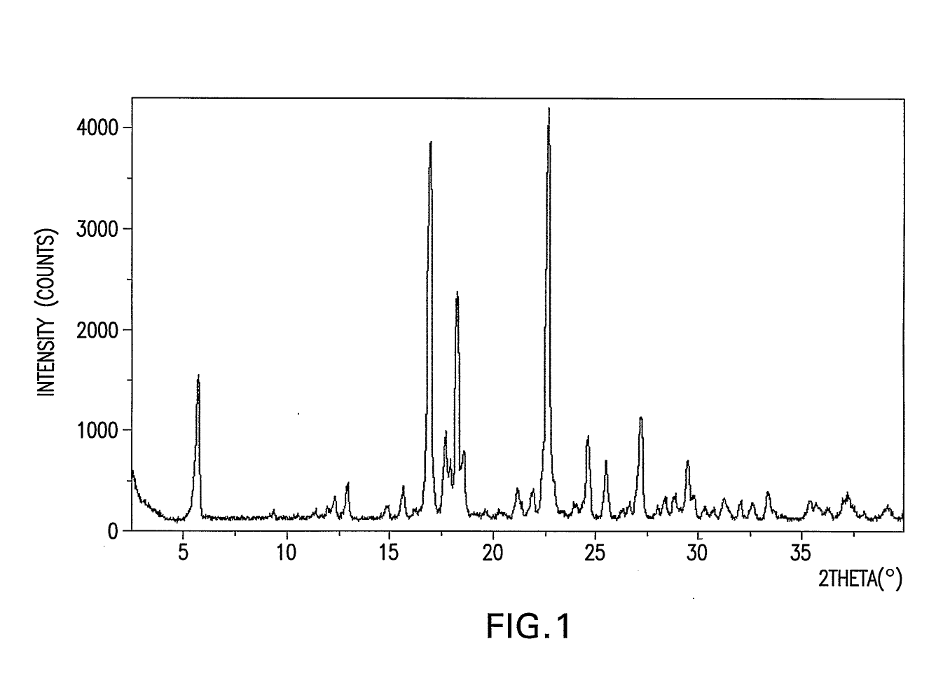

FIG. 1 shows the X-ray diffraction pattern for the crystalline anhydrate form of the L-hydrogen tartrate salt of structural formula II. The crystalline anhydrate exhibited characteristic diffraction peaks corresponding to d-spacings of 3.9, 5.2, and 15.4 angstroms. The crystalline anhydrate was further characterized by the d-spacings of 4.8, 3.3, and 3.0 angstroms. The crystalline anhydrate was even further characterized by the d-spacings of 3.6 and 5.7 angstroms.

In addition to the X-ray powder diffraction patterns described above, the crystalline anhydrate form of the hydrogen tartrate salt of structural formula II was further characterized by its solid-state carbon-13 and fluorine-19 nuclear magnetic resonance (NMR) spectra. The solid-state carbon-13 NMR spectrum was obtained on a Bruker DSX 400WB NMR system using a Bruker 4 mm double resonance CPMAS probe. The carbon-13 NMR spectrum utilized proton/carbon-13 cross-polarization magic-angle spinning with variable-amplitude cross polarization. The sample was spun at 15.0 kHz, and a total of 2048 scans were collected with a recycle delay of 20 seconds. A line broadening of 40 Hz was applied to the spectrum before FT was performed. Chemical shifts are reported on the TMS scale using the carbonyl carbon of glycine (176.03 p.p.m.) as a secondary reference.

The solid-state fluorine-19 NMR spectrum was obtained on a Bruker DSX 400WB NMR system using a Bruker 4 mm CRAMPS probe. The NMR spectrum utilized a simple pulse-acquire pulse program. The samples were spun at 15.0 kHz, and a total of 16 scans were collected with a recycle delay of 30 seconds. A vespel endcap was utilized to minimize fluorine background. A line broadening of 100 Hz was applied to the spectrum before FT was performed. Chemical shifts are reported using poly(tetrafluoroethylene) (teflon) as an external secondary reference which was assigned a chemical shift of −122 ppm.

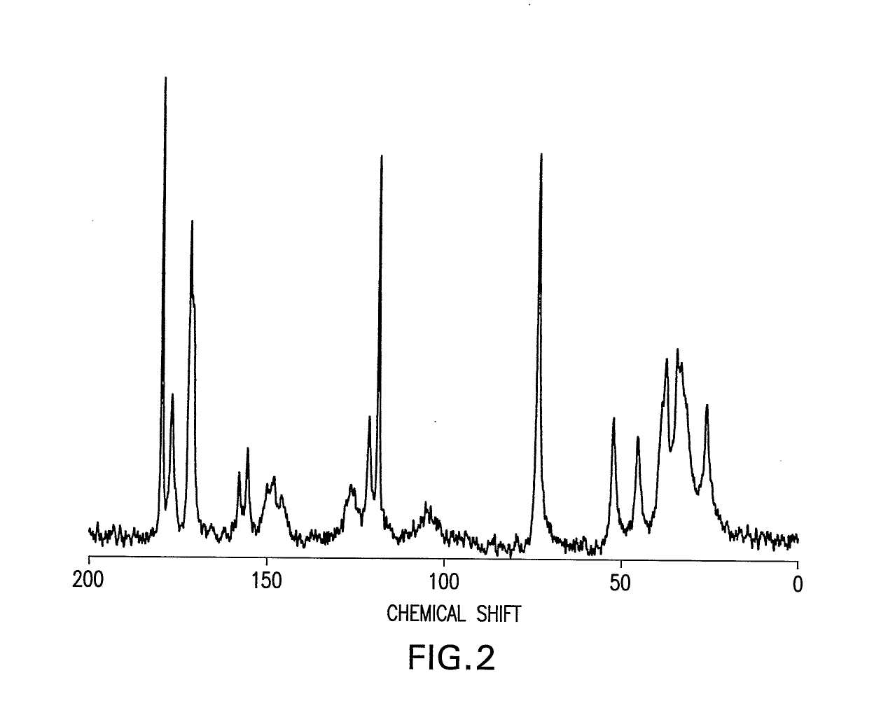

FIG. 2 shows the solid-state carbon-13 CPMAS NMR spectrum for the crystalline anhydrate form of the L-hydrogen tartrate salt of structural formula II. The crystalline anhydrate form exhibited characteristic signals with chemical shift values of 179.8, 121.4, and 45.7 p.p.m. Further characteristic of the crystalline anhydrate form were the signals with chemical shift values of 176.9, 118.8, and 26.3 ppm. Even further characteristic of the crystalline anhydrate form were the signals with chemical shift values of 171.9, 73.8, and 52.5 ppm

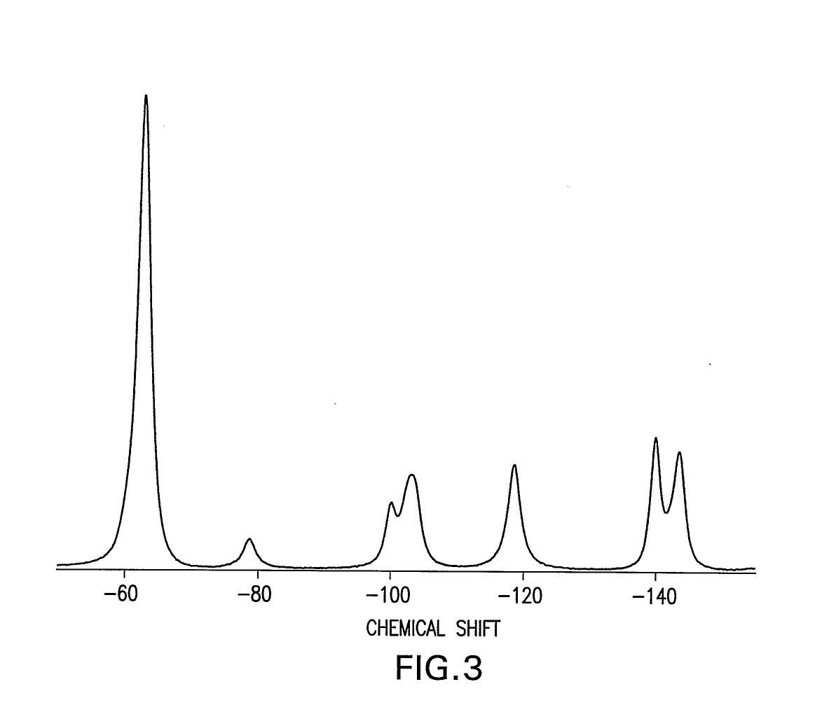

FIG. 3 shows the solid-state fluorine-19 MAS NMR spectrum for the crystalline anhydrate form of the L-hydrogen tartrate salt of structural formula II. The anhydrate form exhibited characteristic signals with chemical shift values of −62.7, −140.0, and −143.6 p.p.m.

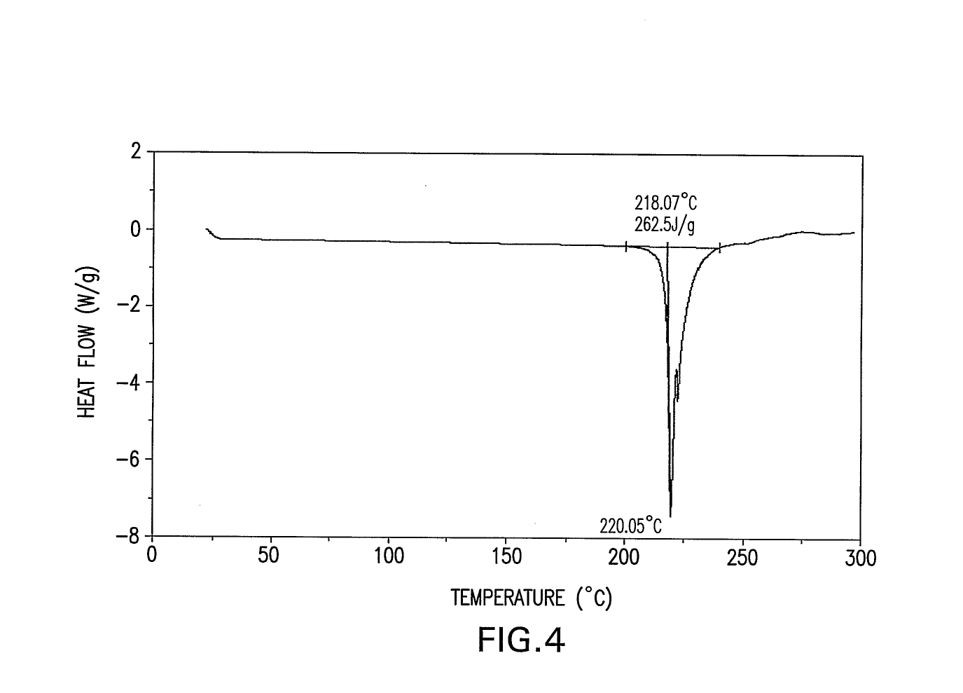

FIG. 4 shows the characteristic DSC curve for the crystalline anhydrate form of the L-hydrogen tartrate salt of structural formula III. A TA Instruments DSC 2910 or equivalent instrumentation was used. Between 2 and 6 mg sample was weighed into an open pan. This pan was then crimped and placed at the sample position in the calorimeter cell. An empty pan was placed at the reference position. The calorimeter cell was closed and a flow of nitrogen was passed through the cell. The heating program was set to heat the sample at a heating rate of 10° C./min to a temperature of approximately 300° C. The heating program was started. When the run was completed, the data were analyzed using the DSC analysis program contained in the system software. The melting endotherm was integrated between baseline temperature points that are above and below the temperature range over which the endotherm was observed. The data reported are the onset temperature, peak temperature, and enthalpy. The DSC curve exhibited a sharp endotherm with a peak temperature of 220.1° C., an extrapolated onset temperature of 218.1° C., and an enthalpy of 262.5 J/g.

The crystalline L-hydrogen tartrate salt anhydrate has a phase purity of at least about 5% of the form with the above X-ray powder diffraction, fluorine-19 MAS NMR, carbon-13 CPMAS NMR, and DSC physical characteristics. In one embodiment the phase purity is at least about 10% of the form with the above solid-state physical characteristics. In a second embodiment the phase purity is at least about 25% of the form with the above solid-state physical characteristics. In a third embodiment the phase purity is at least about 50% of the form with the above solid-state physical characteristics. In a fourth embodiment the phase purity is at least about 75% of the form with the above solid-state physical characteristics. In a fifth embodiment the phase purity is at least about 90% of the form with the above solid-state physical characteristics. In a sixth embodiment the crystalline L-hydrogen tartrate salt anhydrate is the substantially phase pure form with the above solid-state physical characteristics. By the term “phase purity” is meant the solid state purity of the hydrogen tartrate salt anhydrate with regard to a particular crystalline or amorphous form of the salt as determined by the solid-state physical methods described in the present application.

### Examples of Pharmaceutical Formulations

The L-hydrogen tartrate salt of formula II as a crystalline anhydrate can be formulated into a tablet by a direct compression process. A 100 mg potency tablet is composed of 133 mg of the active ingredient, 243 mg mannitol, 20 mg of croscarmellose sodium, and 4 mg of magnesium stearate. The active ingredient, microcrystalline cellulose, and croscarmellose are first blended, and the mixture is then lubricated with magnesium stearate and pressed into tablets.

An intravenous (i.v.) aqueous formulation is prepared by dissolving the L-hydrogen tartrate salt of structural formula II as a crystalline anhydrate in normal saline. For a formulation with a concentration of 5 mg/mL, 6.65 mg of the active ingredient is dissolved in one mL normal saline.

1. A hydrogen tartrate salt of of (3R)-4-[(3R)-3-amino-4-(2,4,5-trifluorophenyl)butanoyl]hexahydro-3-(2,2,2-trifluoroethyl)-2H-1,4-diazepin-2-one of structural formula I:

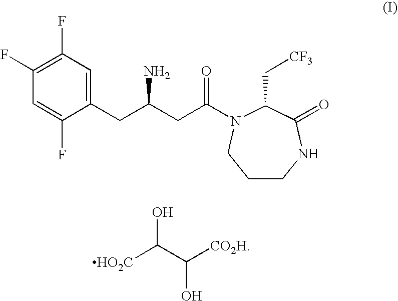

2. The salt of claim 1 wherein said hydrogen tartrate salt is the L-hydrogen tartrate salt of structural formula II:

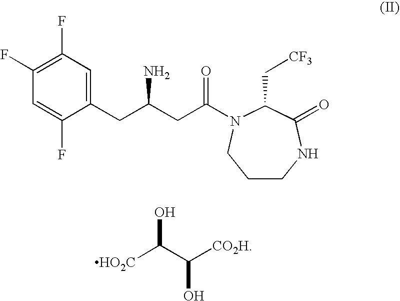

3. The salt of claim 1 wherein said hydrogen tartrate salt is the D-hydrogen tartrate salt of structural formula III:

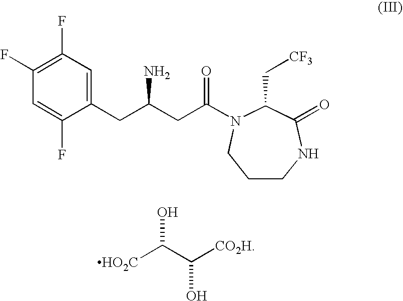

4. The salt of claim 2 characterized in being a crystalline anhydrate.

5. The salt of claim 4 characterized by characteristic absorption bands obtained from the X-ray powder diffraction pattern at spectral d-spacings of 3.9, 5.2, and 15.4 angstroms.

6. The salt of claim 5 further characterized by characteristic absorption bands obtained from the X-ray powder diffraction pattern at spectral d-spacings of 4.8, 3.3, and 3.0 angstroms.

7. The salt of claim 6 further characterized by characteristic absorption bands obtained from the X-ray powder diffraction pattern at spectral d-spacings of 3.6 and 5.7 angstroms.

8. The salt of claim 7 further characterized by the X-ray powder diffraction pattern of FIG. 1.

9. The salt of claim 4 characterized by a solid-state carbon-13 CPMAS nuclear magnetic resonance spectrum showing signals at 179.8, 121.4, and 45.7 ppm.

10. The salt of claim 9 further characterized by a solid-state carbon-13 CPMAS nuclear magnetic resonance spectrum showing signals at 176.9, 118.8, and 26.3 ppm.

11. The salt of claim 10 further characterized by a solid-state carbon-13 CPMAS nuclear magnetic resonance spectrum showing signals at 171.9, 73.8, and 52.5 ppm.

12. The salt of claim 11 further characterized by the solid-state carbon-13 CPMAS nuclear magnetic resonance spectrum of FIG. 2.

13. The salt of claim 4 characterized by a solid-state fluorine-19 MAS nuclear magnetic resonance spectrum showing signals at −62.7, −140.0, and −143.6 ppm.

14. The salt of claim 13 further characterized by the solid-state fluorine-19 MAS nuclear magnetic resonance spectrum of FIG. 3.

15. The salt of claim 4 characterized by the differential scanning calorimetric curve of FIG. 4.

16. A drug substance comprising a detectable amount of the crystalline anhydrate of claim 4.

17. The drug substance of claim 16 comprising about 5% to about 100% by weight of said crystalline anhydrate.

18. The drug substance of claim 16 comprising about 10% to about 100% by weight of said crystalline anhydrate.

19. The drug substance of claim 16 comprising about 25% to about 100% by weight of said crystalline anhydrate.

20. The drug substance of claim 16 comprising about 50% to about 100% by weight of said crystalline anhydrate.

21. The drug substance of claim 16 comprising about 75% to about 100% by weight of said crystalline anhydrate.

22. The drug substance of claim 16 comprising substantially all by weight of said crystalline anhydrate.

23. A pharmaceutical composition comprising a therapeutically effective amount of the salt according to claim 4 in association with one or more pharmaceutically acceptable carriers.

24. A method for the treatment of Type 2 diabetes comprising administering to a patient in need of such treatment a therapeutically effective amount of the salt according to claim 4.

25. (canceled)
